(19) United States
(12) Patent Application Publication
(10) Pub. No.: US 2026/0339766 A1
(43) Pub. Date: Oct. 29, 2026

(54) EQUIPMENT MALFUNCTION PREDICTIVE DIAGNOSIS APPARATUS AND METHOD

(71) Applicants: Hyundai Motor Company, Seoul (KR); Kia Corporation, Seoul (KR); Hyundai AutoEver Corp., Seoul (KR)

(72) Inventors: Ki Won Park, Yongin, Gyeonggi-do (KR); Sung Yong Cho, Incheon (KR); Myoung Gyo Lee, Yongin, Gyeonggi-do (KR); Hyun Jin Kim, Seoul (KR); Byeong Hun Park, Seoul (KR)

(21) Appl. No.: 19/111,692

(22) PCT Filed: Dec. 20, 2022
(86) PCT No.: PCT/KR2022/020803

§ 371 Date: Mar. 13, 2025

(30) Foreign Application Priority Data
Sep. 20, 2022 (KR) 10-2022-0118752

(51) Int. Cl. G06F 11/07; G16H 40/40

(52) U.S. Cl. CPC G06F 11/079; G06F 11/0721; G16H 40/40

(57) ABSTRACT

Proposed are an equipment malfunction predictive diagnosis apparatus and a method thereof, the apparatus including an equipment data collection unit equipped with at least one or more individual boards configured to correspond to types of diagnosis-targeted equipment and have a plurality of input channels into which data of the diagnosis-targeted equipment is input, and an equipment data processing unit provided with a common board configured to supply power to the equipment data collection unit and perform real-time data determination on the diagnosis-targeted equipment's data input from at least one of the plurality of input channels by using a diagnosis model corresponding to a relevant channel, wherein the equipment data collection unit and the equipment data processing unit are connected to each other via a serial communication interface and integrally combined with each other.

### TECHNICAL FIELD

The present disclosure relates to an equipment malfunction predictive diagnosis apparatus and a method thereof, which are configured to realize apparatus miniaturization by integrally combining an equipment data collection unit and an equipment data processing unit with each other, and secure expandability and maintainability of line application.

### BACKGROUND ART

In the field of equipment, prognostics and health management (PHM) is in an increasing trend and being applied for current status diagnosis and malfunction prediction for relevant equipment. Prognostics and health management is growing rapidly to not only reduce costs but also to operate a system stably and realize cost-effective maintenance without downtime.

In addition, unlike centralized data management in a physically separate location where a data center of cloud computing is located, an edge system is used for supporting computing at the edge of a network close to devices and things, enabling each device and thing to analyze and utilize individual data.

Meanwhile, in ordinary equipment malfunction predictive diagnosis apparatuses, it is general that systems for performing edge computing are all separated, a device for collecting data from equipment to be diagnosed and a device for processing the collected data are present individually in order to perform edge computing functions, and connections between two devices use wired communication such as Universal Serial Bus (USB) or Ethernet. However, USB connections are slow, and Ethernet methods also have a problem of data loss. Ultimately, the connection methods between the individual devices have problems with lower maintainability and expandability compared to those when the devices are configured as a single system.

Accordingly, there are requirements for a method to miniaturize a system for performing edge computing into a single device and to transmit data at high speed while preventing data loss and data noise

The foregoing is intended merely to aid in the understanding of the background of the present disclosure, and is not intended to mean that the present disclosure falls within the purview of the related art that is already known to those skilled in the art.

### DISCLOSURE

### Technical Problem

An objective of the present disclosure is to provide an equipment malfunction predictive diagnosis apparatus and a method thereof, wherein costs are reduced by realizing apparatus miniaturization through a structure where an equipment data collection unit and an equipment data processing unit are integrally combined with each other, predictive maintenance is enabled by performing equipment status diagnosis in real-time, and expandability and maintainability of line application are secured.

### Technical Solution

In the present disclosure as a means for solving the above technical problem, there is provided an equipment malfunction predictive diagnosis apparatus, including: an equipment data collection unit equipped with at least one or more individual boards configured to correspond to types of diagnosis-targeted equipment and have a plurality of input channels into which data of the diagnosis-targeted equipment is input; and an equipment data processing unit provided with a common board configured to supply power to the equipment data collection unit and perform real-time data determination on the diagnosis-targeted equipment's data input from at least one of the plurality of input channels by using a diagnosis model corresponding to a relevant channel, wherein the equipment data collection unit and the equipment data processing unit may be connected to each other via a serial communication interface and integrally combined with each other.

For example, the equipment data processing unit may transmit the collected data to an external server, and the external server may include a Prognostics and Health Management (PHM) server configured to determine the data by way of performing status diagnosis of the equipment on the basis of the processed data, predicting a malfunction or a service life of the equipment, and calculating a replacement time of the equipment.

For example, the external server may store a plurality of diagnosis models corresponding to at least one of equipment status-diagnosing and service life predicting algorithms, and the equipment data processing unit may receive the diagnosis model corresponding to a type of the equipment recognized by the common board from the external server.

For example, the equipment data collection unit may convert the data input through the at least one of the plurality of input channels into a digital signal, parse the converted data, and store the parsed data in buffers.

For example, the buffers may include: a first buffer for storing the parsed data parsed at a first sampling rate; and a second buffer for storing the parsed data down-sampled at a second sampling rate lower than the first sampling rate.

For example, the equipment data processing unit may transmit the data from the first buffer to the external server and stores the data from the second buffer in a database.

For example, the data input from the diagnosis-targeted equipment may include at least one of speed data, vibration data, electric current data, and temperature data, which are obtained from the equipment.

For example, the at least one or more individual boards may be provided to be detachable.

For example, when a size of the accumulated data stored in the buffer is larger than a preset size, the equipment data collection unit may insert the data into a data queue so that the data is thread-processed in the equipment data processing unit.

For example, the equipment data processing unit may determine whether a size of the data queue is greater than or equal to zero or not, and extract the data from the data queue in a case where the size of the data queue is greater than or equal to zero.

For example, the serial communication interface may be configured to perform SPI communication.

In the present disclosure as a method for solving the above technical problem, there is provided an equipment malfunction predictive diagnosis method for controlling an equipment malfunction predictive diagnosis apparatus in which an equipment data processing unit and an equipment data collection unit equipped with at least one or more individual boards having a plurality of input channels are connected to each other via a serial communication interface and integrally combined with each other, the method including: inputting data of diagnosis-targeted equipment; and
performing real-time data determination on the diagnosis-targeted equipment's data input from the at least one of the plurality of input channels by using a diagnosis model corresponding to a relevant channel.

For example, the method may further include determining the data in a way of performing status diagnosis of the equipment on the basis of the processed data, predicting a malfunction and a service life of the equipment, and calculating a replacement time of the equipment.

For example, the method may further include converting the data input through the at least one of the plurality of input channels into a digital signal, parsing the converted data, and storing the parsed data in buffers.

For example, the method may further include inserting the data into a data queue so that the data is thread-processed in the equipment data processing unit when a size of the accumulated data stored in a buffer is larger than a preset size.

For example, the method may further include determining whether a size of the data queue is greater than or equal to zero or not and extracting the data from the data queue in a case where the size of the data queue is greater than or equal to zero.

### Advantageous Effects

According to the equipment malfunction predictive diagnosis apparatus and the method thereof in the present disclosure, costs are reduced by realizing apparatus miniaturization through the structure where the equipment data collection unit and the equipment data processing unit are integrally combined with each other, predictive maintenance is enabled by performing equipment status diagnosis in real-time, and expandability and maintainability of line application are secured. In addition, design costs are reduced through the integrally combined structure compared to an external connecting structure, and it is configurable for the apparatus to be adjacent to equipment through the miniaturization.

The effects of the present disclosure are not limited to the above-mentioned effects, and other objectives that are not mentioned will be clearly understood by those skilled in the art from the following description.

### DESCRIPTION OF DRAWINGS

FIG. 1 is a view illustrating a multi-port valve device according to the present disclosure.

FIG. 2 is a side cross-sectional view illustrating the multi-port valve device illustrated in FIG. 1.

FIG. 3 is a front cross-sectional view illustrating the multi-port valve device illustrated in FIG. 1.

FIG. 4 is a view related to FIG. 1. FIG. 1 is an external perspective view illustrating an equipment malfunction predictive diagnosis apparatus according to an exemplary embodiment of the present disclosure.

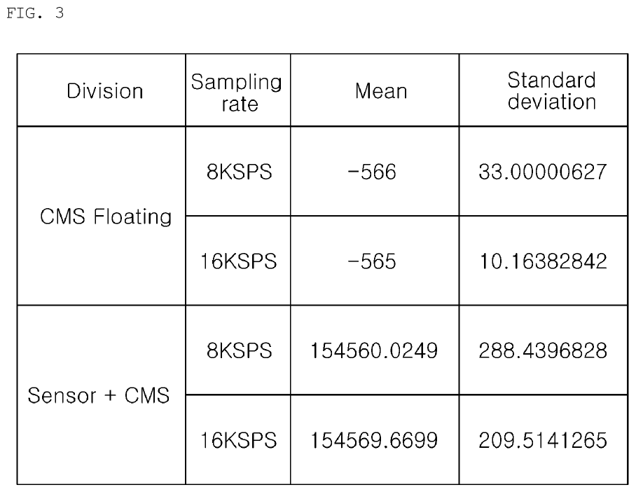

FIG. 2 is a view illustrating a configuration of an equipment data collection unit and an equipment data processing unit that constitute the equipment malfunction predictive diagnosis apparatus.

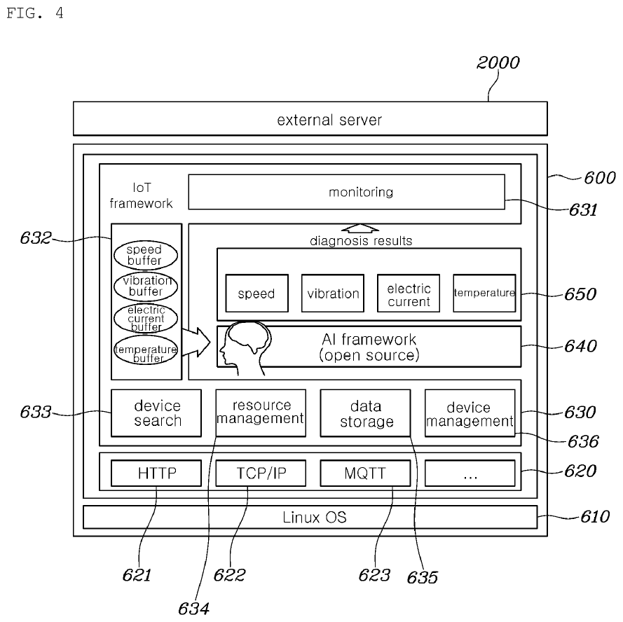

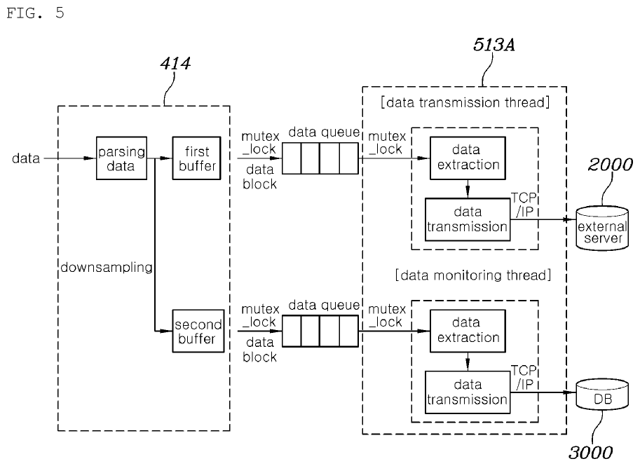

FIG. 3 is a table illustrating reliability verification results of data input through input channels.

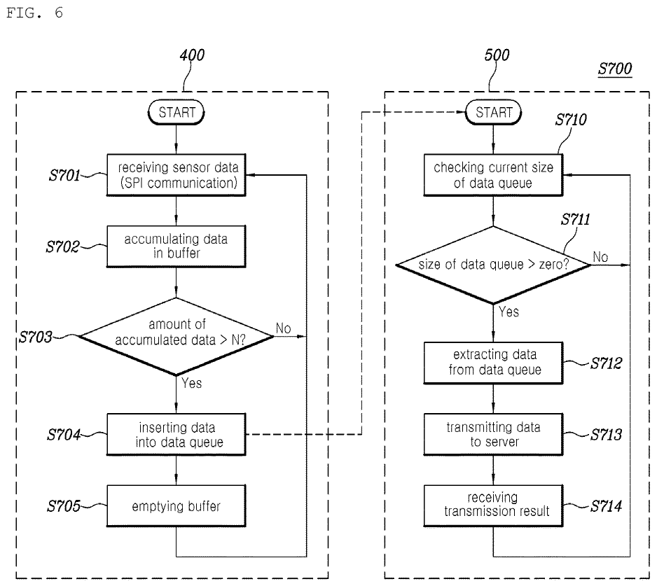

FIG. 4 is a functional block diagram illustrating the entire system.

FIG. 5 is a view illustrating a process in which the equipment data collection unit parses data converted into a digital signal and stores the parsed data in a first buffer and a second buffer.

FIG. 6 is a flowchart illustrating a method of collecting and storing data in an equipment malfunction predictive diagnostic apparatus.

### MODE FOR INVENTIO

Specific structural and functional descriptions of the embodiments of the present disclosure disclosed herein are only for illustrative purposes of the embodiments of the present disclosure. The present disclosure may be embodied in many different forms. Therefore, the embodiments of the present disclosure should not be construed as limiting the present disclosure.

Since the embodiments of the present disclosure may be variously modified in many different forms, specific embodiments will be illustrated in the drawings and described in detail in the specification or application of the present disclosure. However, this is not intended to limit the embodiments in accordance with the concept of the present disclosure to a particular disclosed form. On the contrary, the present disclosure is to be understood to include all various alternatives, equivalents, and substitutes that may be included within the spirit and scope of the present disclosure.

Unless otherwise defined, all terms including technical and scientific terms used herein have the same meaning as commonly understood by those skilled in the art to which this disclosure belongs. It will be further understood that terms, such as those defined in commonly used dictionaries, should be interpreted as having a meaning that is consistent with their meaning in the context of the related art, and will not be interpreted in an idealized or overly formal sense unless expressly so defined herein.

Hereinafter, exemplary embodiments of the present disclosure will be described in detail with reference to the accompanying drawings. Throughout the drawings, the same reference numerals will refer to the same or like parts.

Before describing an equipment malfunction predictive diagnosis method according to the exemplary embodiments of the present disclosure, a configuration of an equipment malfunction predictive diagnosis apparatus 1000 applicable to the exemplary embodiments will first be described.

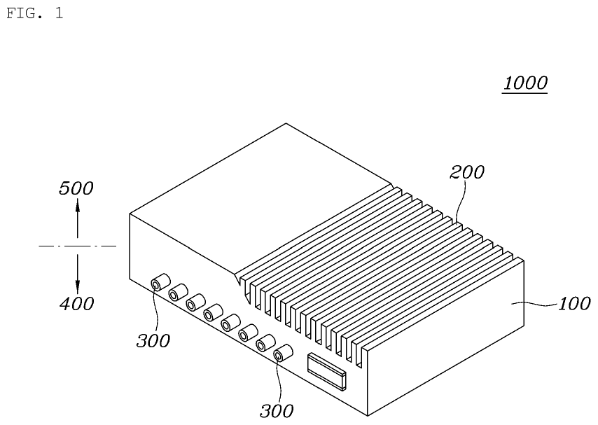

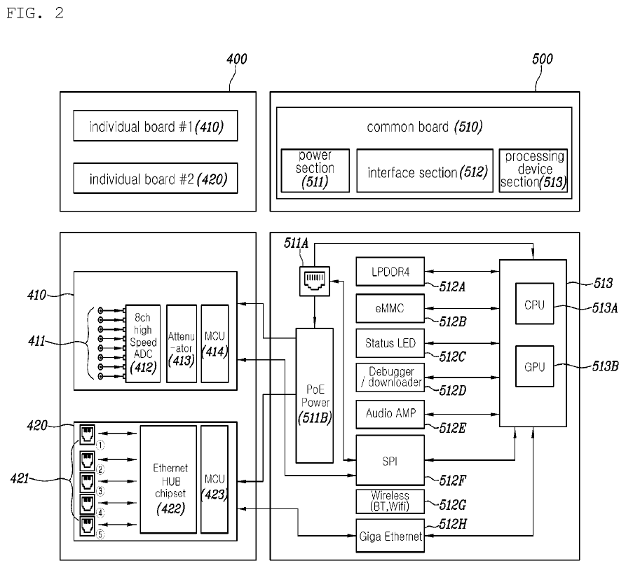

FIG. 1 and FIG. 2 respectively illustrate an external perspective view of an equipment malfunction predictive diagnosis apparatus 1000 and a functional diagram of an equipment data collection unit 400 and an equipment data processing unit 500 that constitute the equipment malfunction predictive diagnosis apparatus 1000 according to the exemplary embodiment of the present disclosure.

FIG. 1 is the external perspective view illustrating the equipment malfunction predictive diagnosis apparatus 1000. The equipment malfunction predictive diagnosis apparatus 1000 is also called a Condition Monitoring System (CMS) module. The equipment malfunction predictive diagnosis apparatus 1000 may include a housing casing 100, cooling fins 200, an equipment data collection unit 400, an equipment data processing unit 500, and external connection ports. First, the housing casing 100 surrounds the exterior of the equipment malfunction predictive diagnosis apparatus 1000 and is formed of aluminum material, thereby reducing the weight of the equipment malfunction predictive diagnosis apparatus 1000. In addition, since the interior and exterior of the equipment malfunction predictive diagnosis apparatus 1000 may be connected to each other by a bolting fastening structure, it is easy to attach and detach individual boards 410 and 420 described below. In addition, a plurality of cooling fins 200 is provided on an outer side of the housing casing 100 to discharge heat generated from a CPU 513A and a GPU 513B.

In addition, the equipment malfunction predictive diagnosis apparatus 1000 may be manufactured in a monolithic structure in which one service is configured in a single large structure compared to a system configuration capable of operating edge computing. The shape of the equipment malfunction predictive diagnosis apparatus 1000 is formed such that a height thereof is low and a width thereof is longer than a front-back length thereof, whereby the center of gravity is lowered, and thus stability may be secured.

Meanwhile, the equipment malfunction predictive diagnosis apparatus 1000 may be provided with external connection ports 300 for connection to the outside. The types of external connection ports 300 include a Bayonet Neill-Concelman (BNC) connector 411 8 ch that is capable of collecting data by connecting a sensor cable thereto, and a Voltage of Common Collector (VCC)/Ground (GND) port, and these may be provided on a front surface of the equipment malfunction predictive diagnosis apparatus 1000. In addition, a power connector, a High-Definition Multimedia Interface (HDMI) connection port, a Universal Serial Bus (USB) connection port, and a Local Area Network (LAN) connection port may be provided on a rear surface of the equipment malfunction predictive diagnosis apparatus 1000. In addition, an LED light capable of indicating status for each input channel and an LED light capable of indicating power supply status may be provided on the front surface of the equipment malfunction predictive diagnosis apparatus 1000. It is preferable that the external connection ports 300 described so far are to be provided on an equipment data collection unit 400 side as shown in FIG. 1 in terms of data input/output.

Referring to FIG. 2, it may be confirmed that a functional block diagram illustrates the equipment data collection unit 400 and the equipment data processing unit 500, which are constituting the equipment malfunction predictive diagnosis apparatus 1000. The equipment data collection unit 400 may be equipped with at least one or more individual boards 410 and 420 corresponding to the types of diagnosis-targeted equipment and having the plurality of input channels. In this case, each of the individual boards 410 and 420 is configured to be detachable so that various types of data may be collected according to the customization of the individual boards 410 and 420.

In addition, a module package that directly collects and controls data may be designed as each of individual boards 410 and 420, and a circuit is configured with a plurality of channels, e. g., eight channels (8 ch), so that data of diagnosis-targeted equipment may be input. The data may be input from the diagnosis-targeted equipment through at least one of the plurality of channels provided in the BNC connector 411 provided in the individual board 410, and the obtained data may be converted from an analog signal to a digital signal through an Analog to Digital Converter (ADC) 412.

In addition, the data converted into the digital signal is transmitted to an attenuator 413 and attenuated according to device management voltage, and the attenuated data may be collected by a Micro Controller Unit (MCU) 414. The collected data may be parsed and stored in a buffer of the controller (i.e., the MCU) 414. Here, data parsing means the task of assembling data and refining this data into desired data.

Meanwhile, the diagnosis-targeted equipment's data input to the plurality of input channels may include at least one of speed data, vibration data, electric current data, and temperature data, which are obtained from sensors provided for each equipment. The types of data described above are exemplary and are not necessarily limited thereto, and any data that may be collected from each equipment is not limited to the types thereof.

A system for ensuring the reliability of diagnosis-targeted equipment′ data input through each input channel is described below. The system for ensuring the reliability may be configured in the form of a single-ended GND by connecting VCC and GND to both the BNC connector 411 and a terminal block of the equipment malfunction predictive diagnosis apparatus 1000 through a power supply. When voltage is applied by way of increasing the voltage from 1 V to 24 V in units of 1 V through the power supply, values output from the ADC 412 may be stored, and an average value of data received 10 times for each of the 8 ch (i.e., the 8 channels) may be calculated.

The results obtained by verifying the reliability through such a configuration are as shown in FIG. 3.

During the verification process, the bit resolution of the equipment malfunction predictive diagnosis apparatus was set to 24 bits so that a value of 2²³ was expressed as a value of the ADC 412 when 24 V was applied.

Specifically, a base voltage is 0.475 V, and in order to check whether a high-speed sampling rate is reproduced or not, the sampling rate was divided into 8 kS/s and 16 kS/s, and the data collected for 10 seconds each was checked. First, in a case when only the equipment malfunction predictive diagnosis apparatus 1000 was present, standard deviations/means of output values of the ADC 412, the output values being floated and expressed, are checked, and in a case when a vibration sensor was attached to the equipment malfunction predictive diagnosis apparatus 1000, standard deviations/means of output values of the ADC 412 are checked, thereby performing comparison for the results.

As shown in FIG. 3, it may be confirmed that the noise generated by the equipment malfunction predictive diagnosis apparatus 1000 itself is negligible through the fact that floating values come in almost constantly, and it may also be confirmed that there is no significant difference in changes between the sampling rates. In the form where the equipment malfunction predictive diagnosis apparatus 1000 is combined with a vibration sensor, it may be confirmed that a ratio of a standard deviation to a mean is 0.19% at 8 kS/s, and a ratio of a standard deviation to a mean is 0.14% at 16 kS/s, thereby showing almost no deviation. As a result, it may be confirmed that reliability has been secured for the data collected by the connection with the vibration sensor.

One of the individual boards 410 and 420 may be provided with an Ethernet module 420. The controller (MCU) 414 of the Ethernet module may also be supplied with power in a way of using POE 511B and may exchange data with Giga Ethernet 512H. In addition, the controller (MCU) 414 may be connected to a plurality of Ethernet connectors via an Ethernet hub chipset. Each Ethernet connector may be connected to an external device via an Ethernet cable.

Referring back to FIG. 2, the equipment data processing unit 500 may be provided with a common board 510 for performing real-time data determination on the diagnosis-targeted equipment's data input from at least one of the plurality of input channels by using a diagnosis model corresponding to a relevant channel. Here, the common board 510 may be configured with a power section 511, an interface section 512, and a processing device section 513.

Specifically, by connecting an RJ-45 standard connector port 511A to the power section 511, power generated through Power of Ethernet (POE) 511B may be allowed to be transmitted to the individual boards 410 and 420 of the equipment data collection unit 400. In addition, the power section 511 may supply power to the equipment data collection unit 400 and also supply power to the processing device section 513 in a form of 1.1 V, 1.5 V, 1.8 V, and 3.3 V. In this case, the form of supplying power is not limited to the form described above. In addition, the interface section 512 may be configured to be connected to the processing device section 513 through LPDDR4 512A and a DDR 32-bit interface. In addition, the interface section 512 may be configured with an integrated package of flash memory and controller and connected to the processing device section 513 through an Embedded Multi-Media Controller (eMMC) 512B and an MMC interface.

A status LED 512C that provides a data collection status alarm for the power or channel section may be connected via General-Purpose Input/Output (GPIO), and a debugger/downloader 512D for firmware may be connected to the processing device section 513 via a USB and a Universal Asynchronous Receiver/Transmitter (UART).

In addition, an Audio AMP 512E for giving a bad alarm after data determination is performed may be connected to the processing device section 513 in an Integrated Interchip Sound (I2S) manner. A connection between the controller (MCU) 414 and the CPU 513A within the equipment data collection unit 400 may be made through SPI communication 512F that will be described below, and may be interfaced in a Reduced Gigabit Media Independent Interface (RGMII) manner. Meanwhile, a wireless mode supports Bluetooth and WiFi 512G, and Giga Ethernet 512H may be configured for an Ethernet module.

In addition, the CPU 513A and the GPU 513B constitute the processing device section 513. The CPU 513A and the GPU 513B may command data collection and preprocessing in real time, and be loaded with Artificial Intelligence (AI)-driven algorithms, thereby being allowed to perform edge computing through real-time data determination.

In addition, the equipment data processing unit 500 may be connected to the equipment data collection unit 400 via a serial communication interface. In order to transmit and process data at high speed without data loss or noise, it is preferable to use the serial communication interface, which is a short-distance communication. For example, serial communication includes Serial Peripheral Interface (SPI) communication 512F. The equipment data processing unit 500 may also consider a method such as Ethernet to connect to the equipment data collection unit 400, but the Ethernet method is not preferable due to a risk of data loss during data transmission. In contrast, the SPI communication 512F has an advantage of being able to transmit data in real time at high speed over short distances and of having no data loss. Accordingly, by using the SPI communication 512F, high-speed data processing is applicable, and data determination may be performed in real time in the equipment data processing unit 500 through the AI-driven algorithms. Accordingly, it may be said that the SPI communication 512F method is a communication method suitable for the equipment malfunction predictive diagnosis apparatus 1000 in which the equipment data collection unit 400 and the equipment data processing unit 500 are integrally combined with each other and configured into the single apparatus.

FIG. 4 is a functional block diagram 600 illustrating the entire system.

Referring to FIG. 4, a functional block diagram 600 for the CPU 513A may include a Linux-based operating system 610, communication protocols 620, and an IoT framework 630 that is an embedded SW. The communication protocols 620 include HyperText Transfer Protocol (HTTP) 621 that is a protocol for hypertext transmission, Transmission Control Protocol/Internet Protocol (TCP/IP) 622 that is an Internet information transmission protocol, and Message Queueing Telemetry Transport (MQTT) 623 that is a message queuing protocol, but these are just examples and are not necessarily limited thereto.

Meanwhile, the IOT framework 630 may be composed of a result monitoring part 631, a data collection/transmission part 632, a device search part 633, a resource management part 634, a data storage part 635, and a management part 636.

First, the device search part 633 may search for an external device connected to the equipment malfunction predictive diagnosis apparatus 1000. The resource management part 634 may manage resources stored in the CPU 513A, and the data storage part 635 may store data collected from equipment. In addition, the management part 636 provides information on which diagnosis model to load. In addition, the resource management part 634 may perform a function of providing a place to store some data within the apparatus and of managing resources by checking occupancy rates of the CPU 513A and the GPU 513B within the apparatus. In the data collection/transmission part 632, data collected from the equipment through the BNC connector 411 is received through each channel and may be collected and transmitted in real time through an internal buffer of the CPU 513A via an internal buffer of the controller (MCU) 414 of the equipment data collection unit 400. The diagnosis model loading and determination part 650 may perform a function of loading a diagnosis model corresponding to a type of equipment recognized in the common board 510, the diagnosis model being provided from an external server 2000, onto a virtual machine of the AI framework 640, so as to provide different diagnosis models by channel. This enables real-time data determination of the pieces of transmitted data, and allows edge computing functions to be performed while visualizing results monitored in the result monitoring part 631 together with the data.

In addition, the external server 2000 may include a Prognostics and Health Management (PHM) server capable of performing a status diagnosis of equipment on the basis of the processed data, predicting a malfunction or service life of the equipment, and calculating a replacement time of the equipment. The external server 2000 may perform predictive maintenance of the equipment on the basis of the data transmitted from the equipment data processing unit 500. In this case, a plurality of diagnosis models corresponding to at least one of equipment status-diagnosing and service life-predicting algorithms may be stored in the external server 2000 and be provided to the equipment data processing unit 500. Accordingly, the equipment data processing unit 500 may receive a diagnosis model corresponding to a type of equipment recognized in the common board 510 among the stored diagnosis models and be able to perform data determination in real time.

FIG. 5 is a view illustrating a process in which the equipment data collection unit 400 parses data converted into a digital signal and stores the parsed data in a first buffer and a second buffer.

Referring to a left side drawing in FIG. 5, it is possible to confirm a process in which data of the equipment data collection unit 400 is split and stored in a first buffer and a second buffer. The equipment malfunction predictive diagnosis apparatus 1000 capable of driving a high-speed sampling rate may receive and parse up to 16,000 samples per second from the BNC connector 411. In order to receive all the parsed data without omission, it is required to store the data primarily by placing an intermediate buffer and control data transmission through a message queue manner. In this case, the data may be split and stored in two buffers, thereby being split and transmitted to a data transmission thread and a data monitoring thread. In order to be transmitted to the data transmission thread, the parsed data parsed at a first sampling rate, which is a high-speed sampling rate, may be stored in the first buffer. In addition, in order to be transmitted to the data monitoring thread, the data may be down-sampled to a second sampling rate lower than the first sampling rate and stored in the second buffer so that there is no delay in real-time data determination.

In this case, in order to utilize shared resources (so called threads) of the CPU 513A, a method of accessing data by synchronizing with time control may be used. Mutex is used as a synchronization mechanism provided by Pthread, and the amount of data to be inserted into each data queue may be controlled by stopping or transmitting the data through a mutex_lock command. Each data queue is implemented as a temporary storage through a ring buffer, and data transmission may be done in a first-in, first-out (FIFO) manner.

Referring to the left side drawing in FIG. 5, it is possible to confirm a process in which the equipment data processing unit 500 transmits the data of the first buffer to the external server 2000, and transmits the data of the second buffer to a database so as to be stored therein. More specifically, the respective pieces of data temporarily stored in the first buffer and the second buffer are stored in respective data queues in the first-in, first-out manner by using a queue method, so as to be transmitted to the respective threads, and the mutex lock command may be used to control access to the shared resources so called the threads, thereby controlling data blocking and data transmission. Each thread may also control the resources required for data transmission and extraction by using the mutex lock command independently, thereby preventing data loss. Accordingly, the data extracted from the threads may be transmitted to the external server 2000 or the database (DB) 3000 through the TCP/IP protocol, thereby being manageable.

Based on the configuration of the equipment malfunction predictive diagnosis apparatus 1000 described above, an equipment malfunction predictive diagnosis method S700 according to an exemplary embodiment is described with reference to FIG. 6.

FIG. 6 is a flowchart of S700 illustrating a method for collecting and storing data in an equipment malfunction predictive diagnosis apparatus 1000 in which an equipment data processing unit 500 and an equipment data collection unit 400 equipped with at least one or more individual boards 410 and 420 having a plurality of input channels according to FIG. 1 are connected to each other via a serial communication interface.

First, in step S701, data collected from a BNC connector 411 is converted into a digital signal through an ADC 412, is temporarily stored in a controller (MCU) 414, and then is transmitted to a CPU 513A of a platform module through internal SPI communication 512F. Thereafter, in step S702, a large amount of data parsed at a high speed (i.e., 16 kS/s) by the CPU 513A is parsed, and then stored and accumulated in a first buffer for storing the parsed data parsed at a first sampling rate and a second buffer for storing parsed data down-sampled at a second sampling rate lower than the first sampling rate. The data may be split and stored in two buffers so as to be split and transmitted to a data transmission thread and a data monitoring thread.

In step S703, the equipment data collection unit 400 may determine whether a size of the accumulated data stored in the first buffer is larger than a preset size N in order to check whether the data is at a level that can be stored in the buffer. This is to check the amount of data accumulated in the buffer and see if the amount of data is at the level that can be stored in the buffer. When it is determined that the size of the accumulated data stored in the first buffer is not larger than the preset size N (i.e., No in step S703), additional data is allowed to be received. However, when it is determined that the size of the accumulated data stored in the first buffer is larger than the preset size N (i.e., Yes in step S703), the data may be inserted into a data queue in step S704, so that the data may be thread-processed in the equipment data processing unit 500. In step S705, when all the data is accumulated in the data queue, the first buffer is emptied to receive following data. In steps S701 to S705, the process described so far may be performed by the equipment data collection unit 400.

A process of the equipment data processing unit 500 that transmits data to the external server 2000 and the database (DB) 3000 after the buffer is emptied is described.

In step S710, the equipment data processing unit 500 may check a current size of the data queue and prepare data collection in the CPU 513A. In step S711, it is possible to determine whether to extract data from the data queue or not by determining whether a size of the data queue is greater than or equal to zero. In a case where the size of the data queue is less than zero (i.e., No in step S711), the size of the data queue is continuously checked. However, in a case where the size of the data queue is greater than or equal to zero (i.e., Yes in step S711), the data may be extracted from the data queue in step S712. In this case, when there is data in the data queue, a mutex_lock command may be used simultaneously to perform time-synchronized access control from the data queue and extract the data.

Afterwards, in step S713, the extracted data is transmitted to a server through the TCP/IP protocol. In step S714, the equipment data processing unit 500 receives a data reception result from the external server 2000 and prepares to extract new data from the data queue again. According to the data processing process as described above, the equipment data processing unit 500 may smoothly perform real-time data determination on the data of the diagnosis-targeted equipment by using the diagnosis model corresponding to the relevant channel.

Ultimately, the costs may be reduced by realizing the apparatus miniaturization through the structure in which the equipment data collection unit and the equipment data processing unit are integrally combined with each other, the predictive maintenance may be enabled by performing the equipment status diagnosis in real time through the AI algorithms, and the expandability and maintainability of line application may be secured.

Meanwhile, the above-described present disclosure may be implemented as computer-readable code in a medium on which a program is recorded. The computer-readable medium includes all types of recording devices in which data readable by a computer system is stored. Examples of the computer-readable medium include a Hard Disk Drive (HDD), a Solid State Disk (SSD), a Silicon Disk Drive (SDD), a ROM, a RAM, a CD-ROM, a magnetic tape, a floppy disk, an optical data storage device, etc. Accordingly, the above detailed description should not be construed as restrictive in all respects but as exemplary. The scope of the present disclosure should be determined by a reasonable interpretation of the appended claims, and all modifications within the equivalent scope of the present disclosure are included in the scope of the present disclosure.

### Description of the Reference Numerals in the Drawings

100: housing casing 200: cooling fins
300: external connection port 400: equipment data collection unit
500: equipment data processing unit 410, 420: individual boards
411: BNC connector 412: ADC
413: attenuator 414: controller (MCU)
510: common board 511: power section
511A: connector port 511B: POE
512: interface section 512A: LPDDR4
512B: eMMC 512C: status LED
512D: debugger/downloader 512E: audio AMP
512F: SPI communication 512G: Bluetooth, WiFi
512H: Giga Ethernet 513: processing device section
513A: CPU 513B: GPU
610: operating system 620: communication protocols
621: HTTP 622: TCP/IP
623: MQTT 630: IOT framework
631: result monitoring part 632: data collection/transmission part
633: device search part 634: resource management part
635: data storage part 636: management part
640: AI framework 650: diagnosis model loading and determination part
1000: equipment malfunction predictive diagnosis apparatus 2000: external server
3000: database (DB)

1. An equipment malfunction predictive diagnosis apparatus, comprising:
an equipment data collection unit equipped with at least one or more individual boards configured to correspond to types of diagnosis-targeted equipment and have a plurality of input channels into which data of the diagnosis-targeted equipment is input; and
an equipment data processing unit provided with a common board configured to supply power to the equipment data collection unit and perform real-time data determination on the diagnosis-targeted equipment's data input from at least one of the plurality of input channels by using a diagnosis model corresponding to a relevant channel,
wherein the equipment data collection unit and the equipment data processing unit are connected to each other via a serial communication interface and integrally combined with each other.

2. The apparatus of claim 1, wherein the equipment data processing unit transmits the collected data to an external server, and
the external server comprises a Prognostics and Health Management (PHM) server configured to determine the data by way of performing status diagnosis of the equipment on the basis of the processed data, predicting a malfunction or a service life of the equipment, and calculating a replacement time of the equipment.

3. The apparatus of claim 2, wherein the external server stores a plurality of diagnosis models corresponding to at least one of equipment status-diagnosing and service life predicting algorithms, and
the equipment data processing unit receives the diagnosis model corresponding to a type of the equipment recognized by the common board from the external server.

4. The apparatus of claim 2, wherein the equipment data collection unit converts the data input through the at least one of the plurality of input channels into a digital signal, parses the converted data, and stores the parsed data in buffers.

5. The apparatus of claim 4, wherein the buffers comprise:
a first buffer for storing the parsed data parsed at a first sampling rate; and
a second buffer for storing the parsed data down-sampled at a second sampling rate lower than the first sampling rate.

6. The apparatus of claim 5, wherein the equipment data processing unit transmits the data from the first buffer to the external server and stores the data from the second buffer in a database.

7. The apparatus of claim 1, wherein the data input from the diagnosis-targeted equipment comprises at least one of speed data, vibration data, electric current data, and temperature data, which are obtained from the equipment.

8. The apparatus of claim 1, wherein the at least one or more individual boards are provided to be detachable.

9. The apparatus of claim 4, wherein, when a size of data stored and accumulated in the buffer is larger than a preset size, the equipment data collection unit inserts the data into a data queue so that the data is thread-processed in the equipment data processing unit.

10. The apparatus of claim 9, wherein the equipment data processing unit determines whether a size of the data queue is greater than or equal to zero or not, and extracts the data from the data queue in a case where the size of the data queue is greater than or equal to zero.

11. The apparatus of claim 1, wherein the serial communication interface is configured to preform SPI communication.

12. An equipment malfunction predictive diagnosis method for controlling an equipment malfunction predictive diagnosis apparatus in which an equipment data processing unit and an equipment data collection unit equipped with at least one or more individual boards having a plurality of input channels are connected to each other via a serial communication interface and integrally combined with each other, the method comprising:
inputting data of diagnosis-targeted equipment; and
performing real-time data determination on the diagnosis-targeted equipment's data input from the at least one of the plurality of input channels by using a diagnosis model corresponding to a relevant channel.

13. The method of claim 12, further comprising:
determining the data in a way of performing status diagnosis of the equipment on the basis of the processed data, predicting a malfunction and a service life of the equipment, and calculating a replacement time of the equipment.

14. The method of claim 12, further comprising:
converting the data input through the at least one of the plurality of input channels into a digital signal, parsing the converted data, and storing the parsed data in buffers.

15. The method of claim 14, further comprising:
inserting the data into a data queue so that the data is thread-processed in the equipment data processing unit when a size of data stored and accumulated in a buffer is larger than a preset size.

16. The method of claim 15, further comprising:
determining whether a size of the data queue is greater than or equal to zero or not and extracting the data from the data queue in a case where the size of the data queue is greater than or equal to zero.
